(19) United States
(12) Patent Application Publication
(10) Pub. No.: US 2026/0249025 A1
(43) Pub. Date: Aug. 27, 2026

(54) POWDER FEEDER, ENDOSCOPE, AND METHOD FOR USING POWDER FEEDER

(71) Applicant: NISSHA CO., LTD., Kyoto-shi, Kyoto (JP)

(72) Inventors: Chuzo TANIGUCHI, Otsu-shi, Shiga (JP); Toshihide YAMAMOTO, Kyoto-shi, Kyoto (JP); Hitoshi HIRAI, Kameoka-shi, Kyoto (JP); Takahiro OKABE, Otsu-shi, Shiga (JP)

(21) Appl. No.: 19/163,558

(22) PCT Filed: Feb. 6, 2024
(86) PCT No.: PCT/JP2024/003865

§ 371 Date: Sep. 9, 2025

(30) Foreign Application Priority Data
Mar. 10, 2023 (JP) 2023-037497

(51) Int. Cl. A61M 13/00; A61B 1/015; A61B 1/07

(52) U.S. Cl. CPC A61M 13/00; A61B 1/015; A61B 1/07

(57) ABSTRACT

A powder feeder is to be attached to a distal end portion of an insertion portion of an endoscope main body to be inserted into a living body and that supplies powder into the living body. The powder feeder includes an attachment portion configured to be attached to the distal end portion of the insertion portion, an air supply portion configured to take in a gas from an air supply nozzle of the endoscope main body, an accommodation portion communicating with the air supply portion and configured to accommodate the powder, a diffusion portion communicating with the accommodation portion and configured to diffuse the powder accommodated in the accommodation portion into the gas fed from the air supply nozzle, and a discharge portion communicating with the diffusion portion and configured to discharge the gas into which the powder has been diffused into the living body together with the powder.

### CROSS REFERENCES TO RELATED APPLICATIONS

This application is a U.S. National stage of International Application No. PCT/JP2024/003865, filed on Feb. 6, 2024. This application claims priority to Japanese Patent Application No. 2023-037497 filed on Mar. 10, 2023 with Japan Patent Office.

### BACKGROUND

### Field of the Invention

The present invention relates to a powder feeder to be attached to an endoscope, an endoscope to which the powder feeder is attached, and a method for using the powder feeder.

### Background Information

In a surgical operation or an endoscopic operation of a living body, an adhesion-preventing material, a hemostatic material, and a covering material, which are powdery materials, may be dispersed to an affected area through an endoscope.

For example, Patent Literature 1 (JP 2809976 B2) discloses dispersing a powdery hemostatic material from an extracorporeal powder diffuser to an affected area using an endoscope. The method for diffusing a powdery hemostatic material disclosed in Patent Literature 1 includes: delivering a catheter to the affected area through an endoscope; sending the hemostatic material from the extracorporeal powder diffuser to the affected area through the catheter together with air; and diffusing the hemostatic material to the affected area.

### SUMMARY

For example, the powdery adhesion-preventing material or the powdery hemostatic material has a property of being gelled on the surface of the affected area by moisture. According to the method for introducing a hemostatic material described in Patent Literature 1, the hemostatic material is solidified in a supply tube or a distal end nozzle such as a catheter for feeding powder to an affected area or causes clogging of the supply tube or the distal end nozzle, and this makes it difficult to deliver a sufficient amount of the hemostatic material to the affected area. In order to prevent such clogging and solidification, air is constantly ejected from a powder diffuser, a double tube is used to prevent moisture from entering the tube, or petrolatum or the like is applied to the distal end.

In addition, the introduction method described in Patent Literature 1 has a problem that the powder spreads over a wide area outside the affected area at the time of ejection from the nozzle. When the air supply pressure is increased to prevent clogging, a tendency in which the powder spreads is significantly observed. The outer diameter of an insertion portion of an endoscope main body is, for example, about 5 mm to 15 mm. A tube for endoscopic surgery passing through the insertion portion is thin with a tube outer diameter of, for example, about 1.7 mm to 4.0 mm and long with a length of about 2 m. Therefore, the air supply pressure to the tube tends to increase, so that the powder scatters in a wide range by the release of the pressure when being ejected from the nozzle. Thus, it becomes difficult to disperse the powder such as a hemostatic material only to the affected area. As described above, when powder scatters outside the affected area at the time of diffusing the powder, the following problems arise such as the adhesion of a large amount of powder to an area other than the affected area, insufficient supply of the powder to the affected area, and the generation of loss of powder.

The present disclosure addresses the problem of sufficiently supplying powder to a specific part and reducing the loss of powder when the powder is introduced into the living body with an endoscope.

Hereinafter, a plurality of aspects will be described as means for solving the problem. These aspects can be combined in any manner if necessary.

A powder feeder according to one aspect of the present disclosure is a powder feeder that is to be attached to a distal end portion of an insertion portion of an endoscope main body to be inserted into a living body and that supplies powder into the living body, the powder feeder including an attachment portion, an air supply portion, an accommodation portion, a diffusion portion, and a discharge portion. The attachment portion is configured to be attached to the distal end portion of the insertion portion. The air supply portion is configured to take in a gas from an air supply nozzle of the endoscope main body. The accommodation portion communicates with the air supply portion and is configured to accommodate the powder. The diffusion portion communicates with the accommodation portion and is configured to diffuse the powder accommodated in the accommodation portion into the gas fed from the air supply nozzle. The discharge portion communicates with the diffusion portion and is configured to discharge the gas into which the powder has been diffused into the living body together with the powder.

The powder feeder described above generates a powdery fluid in the diffusion portion, thereby being capable of uniformly stabilizing the concentration of the powder and the amount of discharged powder in the gas when sufficiently supplying the powder to a specific part in the living body, and reducing the loss of the powder in the tube from the proximal end to the distal end.

In the powder feeder described above, it is preferable that the air supply portion, the accommodation portion, the diffusion portion, and the discharge portion are disposed at positions where an objective lens and a light guide are exposed at the distal end portion in a state where the attachment portion is attached to the distal end portion. The powder feeder thus configured can obtain visual information with an endoscope to which the powder feeder is attached.

In the powder feeder described above, the accommodation portion, the diffusion portion, and the discharge portion may be defined by an accommodation case communicating with the air supply portion and accommodating the powder inside and a mesh provided in an opening of the accommodation case, and the accommodation case and the mesh may be configured to diffuse the powder accommodated inside into the gas with the gas sent from the air supply portion and to discharge a powdery fluid that contains the powder diffused into the gas through the opening. The powder feeder thus configured can sufficiently supply the powder to a specific part and reduce the loss of the powder with a simple configuration including the air supply portion, the accommodation case, and the mesh.

In the powder feeder described above, the accommodation portion, the diffusion portion, and the discharge portion may be defined by an accommodation case that accommodates the powder inside, and may include a gas inlet provided in a first wall surface of the accommodation case and communicating with the air supply portion and a discharge hole provided in a second wall surface of the accommodation case different from the first wall surface as a powdery fluid outlet, and the accommodation case may be configured to diffuse the powder into the gas with the gas entering through the gas inlet and to discharge a powdery fluid that contains the powder diffused into the gas through the discharge hole. The powder feeder thus configured can uniformly stabilize the concentration of the powder and the amount of discharged powder in the gas by sufficiently supplying the powder to a specific part, and reduce the loss of the powder in the tube from the proximal end to the distal end with a simple configuration including the air supply portion and the accommodation case.

In the powder feeder described above, the accommodation portion and the diffusion portion may define a first chamber that includes a gas inlet communicating with the air supply portion, a powdery fluid outlet communicating with the discharge portion, and a powder accommodation space accommodating the powder, the first chamber diffusing the powder into the gas with the gas blown from the gas inlet, and discharging a powdery fluid that contains the powder diffused into the gas through the powdery fluid outlet, and the discharge portion may include a discharge port facing an inside of the living body, a powdery fluid inlet communicating with the powdery fluid outlet, and a second chamber located between the discharge port and the powdery fluid inlet and smaller in volume than the powder accommodation space. The powder feeder thus configured can sufficiently supply the powder to a specific part and can reduce the loss of the powder with a simple configuration including the first chamber functioning as the accommodation portion and the diffusion portion and the second chamber functioning as the discharge portion.

The powder feeder described above may further include a vibrating device that vibrates the powder accommodated in the accommodation portion. The powder feeder thus configured can diffuse the powder while vibrating the powder. Thus, the powder feeder can prevent the aggregation of the powder and the adhesion of the powder on the side surface of the container as compared with the case where the powder is not vibrated, whereby the powder feeder can diffuse the powder so as to reduce the uneven distribution of the powder in the gas.

In the powder feeder described above, the accommodation portion may include a first accommodation portion that accommodates a first powder and a second accommodation portion that accommodates a second powder, the air supply portion may be configured to be able to supply the gas to the first accommodation portion and the second accommodation portion individually and independently, and the diffusion portion and the discharge portion may be configured to discharge a first powdery fluid obtained by diffusing the first powder into the gas when the gas is supplied to the first accommodation portion, and may be configured to discharge a second powdery fluid obtained by diffusing the second powder into the gas when the gas is supplied to the second accommodation portion. The powder feeder thus configured uses the first powder and the second powder different from each other, thereby being capable of discharging a plurality of types of powder. In addition, the powder feeder thus configured can discharge the first powder and the second powder at different timings.

An endoscope according to one aspect of the present disclosure includes an endoscope main body including an insertion portion to be inserted into a living body, a powder feeder to be attached to a distal end portion of the insertion portion, and an air supply device that sends a gas to the powder feeder. The powder feeder includes: an attachment portion configured to be attached to the distal end portion of the insertion portion; an air supply portion configured to take in the gas from an air supply nozzle of the endoscope main body; an accommodation portion communicating with the air supply portion and configured to accommodate powder; a diffusion portion communicating with the accommodation portion and configured to diffuse the powder accommodated in the accommodation portion into the gas fed from the air supply nozzle; and a discharge portion communicating with the diffusion portion and configured to discharge the gas into which the powder has been diffused into the living body together with the powder. The air supply device applies vibration to the powder in the powder feeder due to a change in pressure of the gas when discharging the powder from the powder feeder.

In the endoscope configured as described above, the air supply device applies vibration to the powder in the powder feeder due to a change in the pressure of the gas, and as a result, the powder to be discharged from the powder feeder is easily diffused.

A method for using a powder feeder according to one aspect of the present disclosure includes a replacement step, an insertion step, and a powdery fluid discharge step. The replacement step includes inserting an insertion portion of an endoscope into a living body, extracting the insertion portion inserted into the living body from the living body, replacing a hood at a distal end portion of the insertion portion with a powder feeder accommodating powder, and connecting the powder feeder to a tube for air supply. The reinsertion step includes inserting the insertion portion to which the powder feeder is attached toward a specific part in the living body. The powdery fluid discharge step includes sending a gas to the powder feeder located in the living body, and discharging the powder accommodated in the powder feeder to the specific part as a powdery fluid. According to such a method for using a powder feeder, the powder can be delivered to the vicinity of the specific part in the state of being accommodated in the powder feeder. Further, a powdery fluid can be generated in the living body by the diffusion portion, whereby the concentration of the powder and an amount of the discharged powder in the gas can be uniformly stabilized when the powder is sufficiently supplied to the specific part in the living body, and the loss of the powder in the tube from the proximal end to the distal end can be reduced.

The powder feeder or the endoscope according to the present disclosure can sufficiently supply powder to a specific part and reduce the loss of the powder when introducing the powder into the living body by the endoscope. The method for using a powder feeder according to the present disclosure is a method suitable for the powder feeder according to the present disclosure, and can sufficiently bring out the effect of reducing the loss of the powder in the powder feeder according to the present disclosure.

### BRIEF DESCRIPTION OF THE DRAWINGS

FIG. 1 is a perspective view illustrating an example of an endoscope main body.

FIG. 2 is a partially enlarged perspective view of a distal end portion of the endoscope main body to which a hood is attached.

FIG. 3 is a partially enlarged perspective view of the distal end portion of the endoscope main body to which the hood is attached.

FIG. 4 is a perspective view illustrating an appearance of a hood body.

FIG. 5 is a block diagram for describing a device to be connected to the endoscope main body.

FIG. 6 is a partially enlarged perspective view of the distal end portion of the endoscope main body to which a powder feeder is attached.

FIG. 7 is a partially enlarged perspective view for describing a method for attaching the powder feeder to the distal end portion of the endoscope main body.

FIG. 8 is a schematic diagram illustrating an example of configurations of an air supply portion, an accommodation portion, a diffusion portion, and a discharge portion of the powder feeder according to a first embodiment.

FIG. 9 is a schematic diagram illustrating another example of configurations of the air supply portion, the accommodation portion, the diffusion portion, and the discharge portion of the powder feeder according to the first embodiment.

FIG. 10 is a schematic diagram illustrating another example of configurations of the air supply portion, the accommodation portion, the diffusion portion, and the discharge portion of the powder feeder according to the first embodiment.

FIG. 11 is a schematic diagram illustrating another example of configurations of the air supply portion, the accommodation portion, the diffusion portion, and the discharge portion of the powder feeder according to the first embodiment.

FIG. 12 is a schematic diagram illustrating another example of configurations of the air supply portion, the accommodation portion, the diffusion portion, and the discharge portion of the powder feeder according to the first embodiment.

FIG. 13 is a schematic diagram illustrating an example of configurations of an air supply portion, an accommodation portion, a diffusion portion, and a discharge portion of a powder feeder according to a second embodiment.

FIG. 14 is a schematic diagram illustrating an example of configurations of an air supply portion, an accommodation case, and a mesh of a powder feeder according to a third embodiment.

FIG. 15 is a schematic diagram for describing a behavior of powder in the accommodation case and the mesh of the powder feeder according to the third embodiment.

FIG. 16 is a schematic diagram illustrating another example of configurations of the air supply portion, the air supply portion, the accommodation case, and the mesh of the powder feeder according to the third embodiment.

FIG. 17 is a schematic diagram illustrating another example of configurations of the air supply portion, the air supply portion, the accommodation case, and the mesh of the powder feeder according to the third embodiment.

FIG. 18 is a schematic diagram for describing clogging of the mesh with powder in the powder feeder according to the third embodiment.

FIG. 19 is a schematic diagram illustrating an example of configurations of an air supply portion, an accommodation case, and a mesh of a powder feeder according to a fourth embodiment.

FIG. 20 is a schematic diagram for describing a behavior of powder in the accommodation case and the mesh of the powder feeder according to the fourth embodiment.

FIG. 21 is a schematic diagram illustrating an example of a configuration of a powder feeder according to a fifth embodiment.

FIG. 22 is a schematic diagram illustrating another example of the configuration of the powder feeder according to the fifth embodiment.

FIG. 23 is a schematic diagram illustrating another example of the configuration of the powder feeder according to the fifth embodiment.

FIG. 24 is a schematic diagram illustrating an example of configurations of an air supply portion, an accommodation portion, a diffusion portion, a discharge portion, and a vibrating device of a powder feeder according to a sixth embodiment.

FIG. 25 is a perspective view illustrating another example of the vibrating device used in the powder feeder according to the sixth embodiment.

FIG. 26 is a timing chart for describing the operation of an air supply device of the powder feeder according to the sixth embodiment.

FIG. 27 is a schematic diagram illustrating an example of configurations of an air supply portion, an accommodation portion, a diffusion portion, and a discharge portion of a powder feeder according to a seventh embodiment.

FIG. 28 is a schematic diagram illustrating an example of configurations of an air supply portion, an accommodation portion, a diffusion portion, and a discharge portion of a powder feeder according to an eighth embodiment.

FIG. 29 is a schematic diagram illustrating an example of a configuration of another powder feeder.

### DETAILED DESCRIPTION OF EMBODIMENTS

### First Embodiment

### (1) Overall Configuration of Endoscope

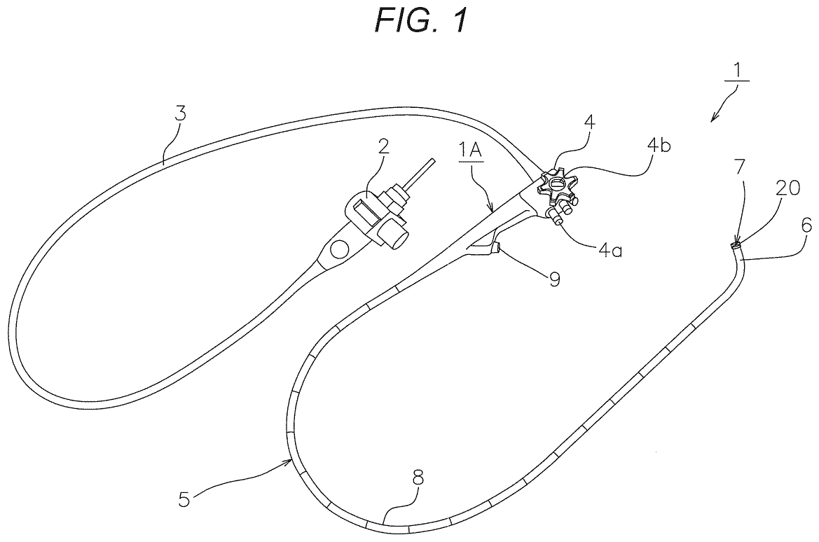

FIG. 1 illustrates an example of an appearance of an endoscope main body 1A of an endoscope 1. The endoscope 1 includes the endoscope main body 1A and a transparent cylindrical hood 20.

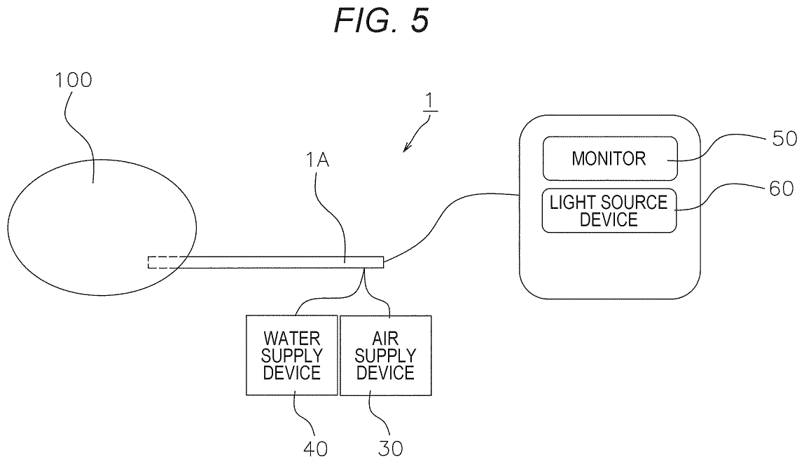

The endoscope main body 1A includes a connection unit 2 and a universal cord 3 for connection with a device. As illustrated in FIG. 5, examples of the device to be connected to the endoscope main body 1A include an air supply device 30 that sends a gas (for example, air, nitrogen, or oxygen) through the universal cord 3, a water supply device 40 that sends water through the universal cord 3, a monitor 50 that displays an image captured by the endoscope main body 1A, and a light source device 60 that emits light transmitted through the universal cord 3. The universal cord 3 is provided with a pipeline (not illustrated) for sending water and gas and a signal cable (not illustrated) for transmitting captured image data.

The endoscope main body 1A includes an operation unit 4 for operation and an insertion portion 5 to be inserted into a living body 100 (see FIG. 5). A bending portion 6 and a distal end portion 7 are located at the tip of the insertion portion 5, and the operation unit 4 and the bending portion 6 are connected by a flexible portion 8. The operation unit 4 is provided with buttons 4a such as a suction button, an air/water supply button, and a shutter button. In response to the operation on the button 4a, the endoscope main body 1A performs operations such as sending air, sending water, and suction through the insertion portion 5. In addition, by operating the button 4a related to imaging such as a shutter button, a shutter and illumination can be operated.

The cylindrical hood 20 is attached to the distal end portion 7 of the endoscope main body 1A. Before powder is supplied into the living body 100, the hood 20 is replaced with a powder feeder 70, and the powder feeder 70 is attached to the distal end portion 7 of the insertion portion 5. For example, when the treatment of an affected area in the living body 100 by the endoscope 1 is finished and a powdery hemostatic material (powder) is supplied to the affected area, the insertion portion 5 is extracted from the living body 100, the hood 20 is removed, and the powder feeder 70 is attached. The insertion portion 5 to which the powder feeder 70 is attached is inserted into the living body 100 again.

An angle knob 4b of the operation unit 4 is connected to the distal end portion 7 of the endoscope main body 1A by, for example, a wire (not illustrated). Due to the operation on the angle knob 4b, the endoscope main body 1A can bend the bending portion 6 in various directions (for example, up, down, left, and right), and can direct the distal end portion 7 in various directions. Since the endoscope main body 1A can bend the bending portion 6, the endoscope main body 1A can be used not only to facilitate insertion of the insertion portion 5 into the living body 100 but also to observe the inside of the body cavity at 360 degrees.

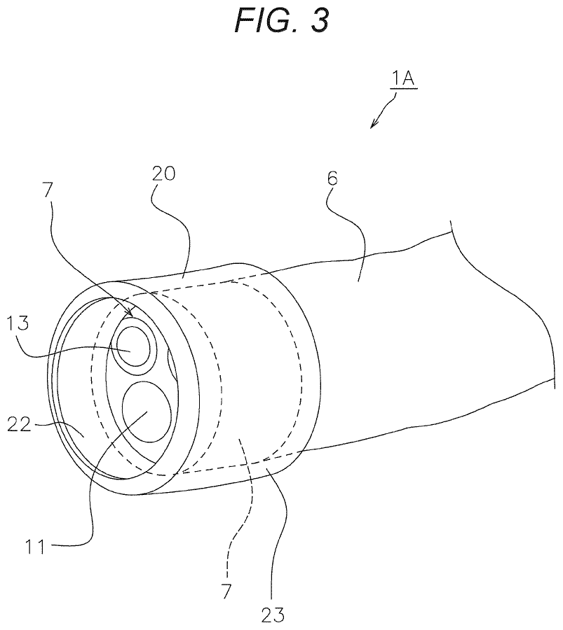

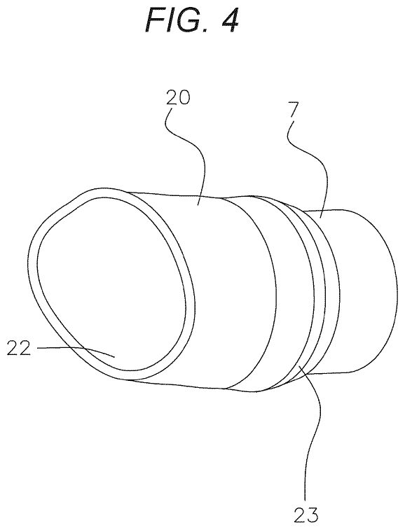

A forceps channel 9 is located near the operation unit 4. The forceps channel 9 is used for inserting and removing a treatment tool. A forceps, for example, that is inserted through the forceps channel 9 can be delivered to the distal end portion 7. The treatment tool is also delivered to the living body 100 using, for example, a guide tube (not illustrated) provided on the surface of the insertion portion 5 in addition to being delivered to the living body 100 through the inside of the insertion portion 5. The treatment tool delivered through the outside of the insertion portion 5 passes through the outside of the hood 20 (see FIGS. 3 and 4) to be described later. The hood 20 is transparent, and thus, the treatment tool passing through the outside of the hood 20 can also be viewed through the hood 20.

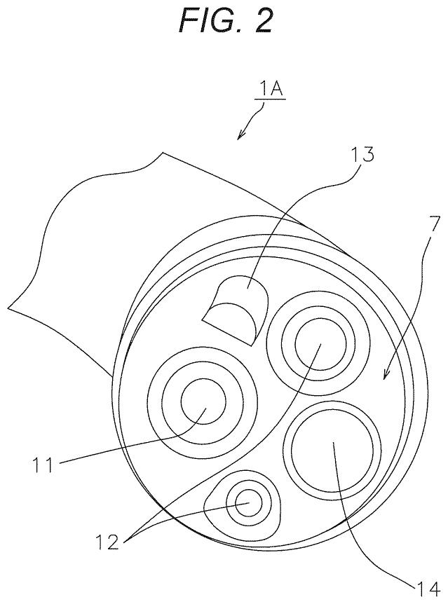

As illustrated in FIG. 2, the distal end portion 7 of the endoscope main body 1A is provided with, for example, an objective lens 11, a light guide lens 12, a tube 13 for air supply and water supply, and a forceps inlet/outlet port 14. A CMOS image sensor (not illustrated) is disposed inside the objective lens 11. An image captured by the CMOS image sensor is displayed on the monitor 50 provided in a device body to which the connection unit 2 is connected.

### (2) Hood

As illustrated in FIG. 3, the hood 20 is attached to the distal end portion 7 of the endoscope main body 1A. The hood 20 is a transparent cylindrical body, and has a configuration in which visual information regarding a body cavity of the living body 100 can be obtained through the hood 20. In order to obtain the transparent hood 20, a transparent polymer material is used as a material of the hood 20. Examples of the transparent polymer material include a transparent resin and a transparent elastomer. Examples of the transparent resin or transparent elastomer used for the hood 20 include polycarbonate and silicone. The hood 20 has a cylindrical shape, and examples of the cross-sectional shape perpendicular to the axis of the cylindrical shape include a circular shape, an elliptical shape, and a quadrangular shape. In the cylindrical hood 20, the size of the cross-sectional shape perpendicular to the axis may be changed along the axis. In other words, the surface of the cylindrical hood 20 may have a frustum shape. In the cylindrical hood 20, the cross-sectional shape perpendicular to the axis may be changed along the axis. For example, the cross-sectional shape of the front part of the hood 20 may be elliptical, and the cross-sectional shape of the rear part may be circular. The front part of the hood 20 may be obliquely cut.

As illustrated in FIGS. 3 and 4, the hood 20 includes a protruding portion 22 protruding from the distal end portion 7 and a mounting portion 23 to be mounted to the distal end portion 7. The protruding portion 22 and the mounting portion 23 of the hood 20 may be formed of different materials. For example, the protruding portion 22 may be formed of polycarbonate, and the mounting portion 23 may be formed of silicone.

The protruding portion 22 of the hood 20 has, for example, an outer diameter of 3 mm to 15 mm and a length of 2 mm to 15 mm. The thickness of the protruding portion 22 is about 0.5 mm to 2 mm.

### (3) Powder Feeder According to First Embodiment

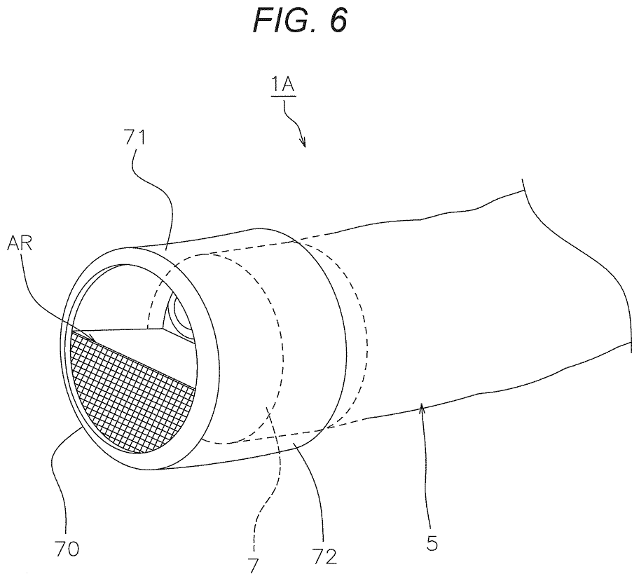

As illustrated in FIG. 6, the powder feeder 70 according to the first embodiment is attached to the distal end portion 7 of the insertion portion 5 of the endoscope main body 1A to be inserted into the living body 100. The powder feeder 70 is a device that feeds powder into the living body 100.

The powder feeder 70 has an attachment portion 72 configured to be attached to the distal end portion 7 of the insertion portion 5. The attachment portion 72 is a section corresponding to the mounting portion 23 of the hood 20, and has the same configuration as, for example, the mounting portion 23 of the hood 20 that has been conventionally used. The attachment portion 72 is an annular section made of, for example, elastomer or plastic. Examples of the elastomer constituting the attachment portion 72 include silicone. Therefore, the hood 20 can be easily replaced with the powder feeder 70 by attaching or removing the powder feeder 70 to or from the distal end portion 7 in the same manner as the manner of attaching or removing the hood 20 to or from the distal end portion 7.

The powder feeder 70 has a main body 71 protruding from the distal end portion 7 like the protruding portion 22 of the hood 20. The main body 71 protrudes from the distal end portion 7 so that a region for accommodating the powder is provided in front of the distal end portion 7. That is, as illustrated in FIG. 6, the configuration of the powder feeder 70 includes the main body 71 and the attachment portion 72.

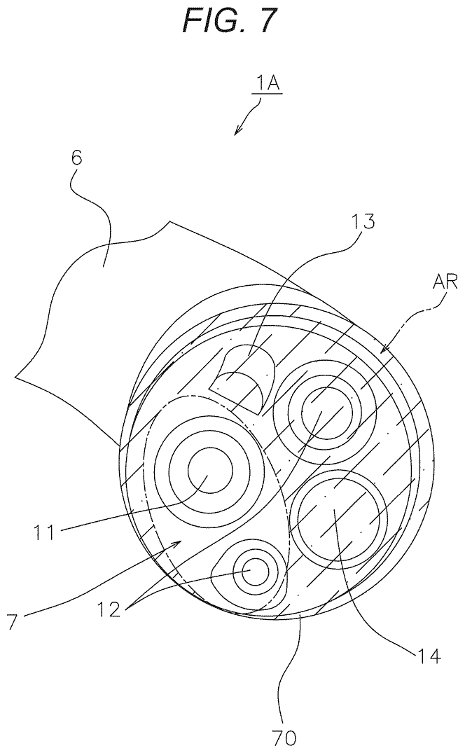

FIG. 7 illustrates an example of a region AR where the powder feeder 70 covers the front surface of the distal end portion 7 by hatching with a dash-dotted line. In order to move the insertion portion 5 in the living body 100, the powder feeder 70 is configured to maintain the front surfaces of the objective lens 11 and the light guide lens 12 open. In the example illustrated in FIG. 7, one of the two light guide lenses 12 is covered with the powder feeder 70. However, the other of the two light guide lenses 12 is exposed, so that the front side of the powder feeder 70 can be illuminated by light emitted from the light guide lens 12. In order to ensure a volume for accommodating the powder at the small distal end portion 7 of the insertion portion 5, it is necessary to ensure as large an occupied space as possible in a space in front of the distal end portion 7, and it is also necessary to make it possible to obtain visual information from the distal end portion 7 at the same time. Note that the region AR where the powder feeder 70 covers the front surface of the distal end portion 7 is not limited to the example illustrated in FIG. 7. For example, the powder feeder 70 may be configured such that the two light guide lenses 12 are both exposed.

### (3-1) First Configuration Example of Powder Feeder

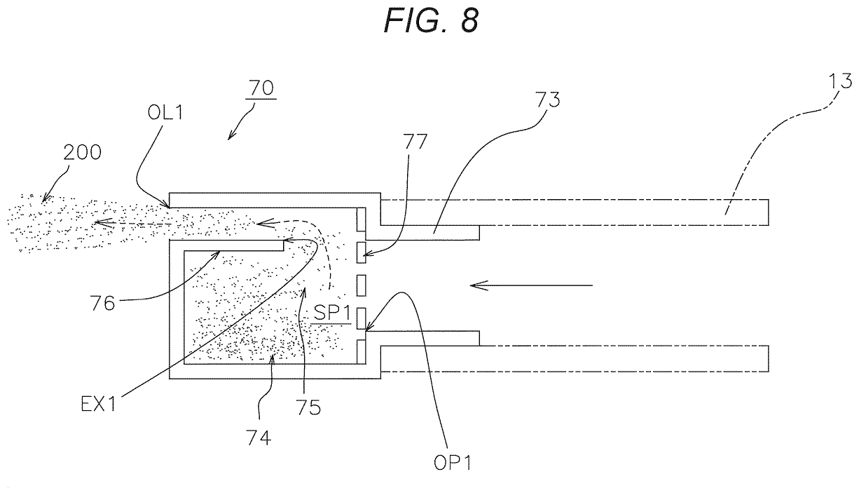

The powder feeder 70 according to the first embodiment includes an air supply portion 73, an accommodation portion 74, a diffusion portion 75, and a discharge portion 76 as illustrated in FIG. 8. The air supply portion 73, the accommodation portion 74, the diffusion portion 75, and the discharge portion 76 are preferably disposed in the region AR indicated by a dash-dotted line in FIG. 7 when viewed in the insertion direction of the distal end portion 7. This is because a portion protruding to the outside of the distal end portion 7 and the main body 71 when viewed in the insertion direction of the distal end portion 7 hinders the insertion. The air supply portion 73, the accommodation portion 74, the diffusion portion 75, and the discharge portion 76 are fixed to the main body 71 of the powder feeder 70. FIG. 8 illustrates a first configuration example of the powder feeder 70. In FIG. 8, a solid arrow indicates a direction in which a gas flows, and a dashed arrow indicates a direction in which a powdery fluid flows. In the drawings described below, solid arrows and dashed arrows similarly indicate directions in which the gas and the powdery fluid flow. In addition, the powder 200 is indicated by an aggregate of a large number of points in FIG. 8. In the drawings described below, the powder is similarly represented by an aggregate of a large number of points.

The discharge portion 76 is a second chamber having an opening to the outside of the powder feeder 70. The second chamber as the discharge portion 76 has, for example, a thin cylindrical shape. The area of the opening of the second chamber is smaller than the cross-sectional area of the tube 13 passing through the insertion portion 5 (the area of the cross-section perpendicular to the gas flowing direction), for example. In such a configuration, the flow velocity of the gas flowing through the second chamber is higher than the flow velocity of the gas flowing through the tube 13. Conversely, when it is desired to make the flow velocity of the gas flowing through the second chamber lower than the flow velocity of the gas flowing through the tube 13, the area of the opening of the second chamber is set larger than the cross-sectional area of the tube 13. The first chamber and the second chamber are surrounded by a resin wall, for example.

The diffusion portion 75 communicating with the discharge portion 76 is disposed upstream of the discharge portion 76. The diffusion portion 75 has a function of diffusing the powder 200 into the gas sent from the tube 13 as an air supply nozzle. For example, in the diffusion portion 75, a turbulent flow is generated, and the powder 200 is dispersed by the turbulent flow. On the other hand, a laminar flow is generated in the tube 13 and the second chamber.

The accommodation portion 74 communicating with the diffusion portion 75 is disposed upstream of the diffusion portion 75. The accommodation portion 74 has a function of accommodating the powder 200. The function of accommodating the powder 200 is a function of keeping the powder 200 in the powder feeder 70 until the powder feeder 70 enters the living body 100 and discharges the powder 200. The accommodation portion 74 and the diffusion portion 75 define a first chamber located upstream of the discharge portion 76 and downstream of the air supply portion 73. In the configuration illustrated in FIG. 8, the second chamber and the first chamber are separated, so that the powder 200 is less likely to spill outside the powder feeder 70. The first chamber and the air supply portion 73 are separated by a mesh 77. The mesh 77 prevents the powder 200 accommodated in the accommodation portion 74 from flowing out of the powder feeder 70 through the air supply portion 73. The first chamber between the discharge portion 76 and the air supply portion 73 can be regarded as also serving as the accommodation portion 74 and the diffusion portion 75.

The air supply portion 73 is disposed upstream of the accommodation portion 74. The mesh 77 is provided between the accommodation portion 74 and the air supply portion 73. The air supply portion 73 communicates with the accommodation portion 74 through the mesh 77. The air supply portion 73 is a section configured to take in a gas from the tube 13 of the endoscope main body 1A. The air supply portion 73 functions as a fitting portion to be fitted into the tube 13, and also has a function of connecting to the tube 13. Therefore, the outer shape of at least a part of the air supply portion 73 coincides with the shape of the flow path of the tube 13. The entire outer shape of the air supply portion 73 in FIG. 8 coincides with the shape of the flow path of the tube 13. In the powder feeder 70 illustrated in FIG. 8, the position of the opening between the air supply portion 73 and the accommodation portion 74 is shifted with respect to the position of the opening of the second chamber which is the discharge portion 76 when viewed in the direction of the flow path of the tube 13. As a result, the airflow flowing from the air supply portion 73 toward the second chamber is bent on the way, so that a turbulent flow is likely to occur.

### (Structure of Powder Feeder)

In addition to the configuration of the powder feeder 70 described above, the structure of the powder feeder 70 will be described a little more. In the powder feeder 70 according to the first configuration example in FIG. 8, the first chamber serves as both the accommodation portion 74 and the diffusion portion 75. Therefore, the first chamber is provided with a clearance in addition to the volume of the powder 200 so as to be capable of diffusing the powder. For example, the first chamber preferably has a volume of, for example, 1.1 times or more the volume of the powder 200 to be accommodated. When the powder 200 is packed into the first chamber without any gaps, it is difficult to diffuse the powder 200 into the gas in the first chamber. An internal space of the first chamber serves as a powder accommodation space SP1 accommodating the powder 200. The accommodation portion 74 and the diffusion portion 75 have a gas inlet OP1 communicating with the air supply portion 73 and a powdery fluid outlet EXI communicating with the discharge portion 76 in addition to the powder accommodation space SP1. In the powder accommodation space SP1 of the first chamber in the powder feeder 70 illustrated in FIG. 8, the powder 200 is diffused into the gas by the gas blown from the gas inlet OP1, and the gas in which the powder 200 is diffused is discharged through the powdery fluid outlet EX1. In the present specification, a fluid obtained by diffusing the powder 200 in a gas is referred to as a powdery fluid.

The discharge portion 76 has a discharge port OL1 facing the inside of the living body 100 and a powdery fluid inlet communicating with the powdery fluid outlet EX1. In the powder feeder 70 illustrated in FIG. 8, the powdery fluid inlet of the discharge portion 76 coincides with the powdery fluid outlet EX1 of the accommodation portion 74 and the diffusion portion 75. However, another chamber may be further provided between the discharge portion 76 and the diffusion portion 75. In that case, the powdery fluid inlet of the discharge portion 76 is provided separately from the powdery fluid outlet EX1. The other chamber is, for example, a chamber having a function of shaking off a lump of the powder 200.

The second chamber serving as the discharge portion 76 has a smaller volume than the powder accommodation space SP1 of the first chamber. The second chamber serving as the discharge portion 76 is a space from the powdery fluid inlet (powdery fluid outlet EX1) of the discharge portion 76 to the discharge port OL1. By setting the volume of the second chamber to be smaller than the volume of the first chamber, the amount of the powder 200 remaining in the discharge portion 76 can be reduced. In addition, this configuration helps limit the discharge direction and the discharge area of the powder 200 by the path from the powdery fluid inlet (powdery fluid outlet EX1) to the discharge port OL1.

### (3-2) Second to Fourth Configuration Examples of Powder Feeder

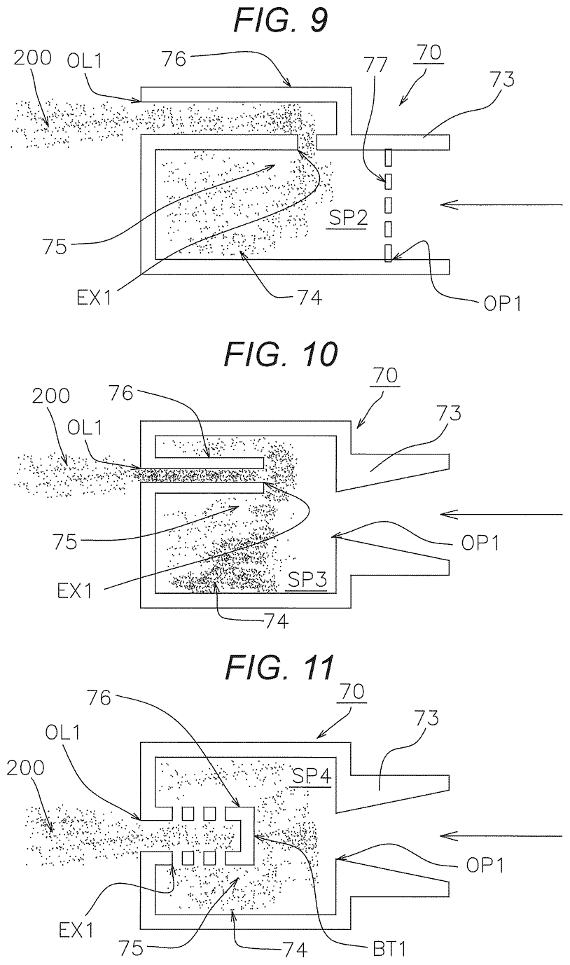

FIG. 9 illustrates a second configuration example of the powder feeder 70. The powder feeder 70 according to the second configuration example illustrated in FIG. 9 is different from the powder feeder 70 according to the first configuration example illustrated in FIG. 8 in the shapes of the air supply portion 73 and a powder accommodation space SP2 and the direction and size of the powdery fluid outlet EX1. The powdery fluid outlet EX1 of the powder feeder 70 according to the second configuration example in FIG. 9 is smaller and is oriented in a direction perpendicular to the flowing direction of the gas through the tube 13. In the powder feeder 70 according to the second configuration example in FIG. 9, the boundary between the air supply portion 73 and the powder accommodation space SP2 is defined by a mesh 77. Therefore, the direction of flow of the gas entering the powder accommodation space SP2 from the air supply portion 73 is affected not only by the difference in the shapes of the air supply portion 73 and the powder accommodation space SP2 but also by the powdery fluid outlet EX1. When the airflow passes through the powdery fluid outlet EX1, the flow velocity of the airflow is higher than that of the airflow through the tube 13. The powder 200 is sucked and diffused by the fast airflow passing through the powdery fluid outlet EX1 and discharged from the discharge portion 76.

FIG. 10 illustrates a third configuration example of the powder feeder 70. The powder feeder 70 according to the third configuration example in FIG. 10 is different from the powder feeder 70 in FIG. 8 in the arrangement position and shape of the discharge portion 76, the presence or absence of the mesh 77, and the size of the gas inlet OP1. The discharge portion 76 in FIG. 10 is a thin cylindrical discharge tube. The discharge tube protrudes into a powder accommodation space SP3 of the third configuration example in FIG. 10. The discharge through the discharge tube makes it difficult for the powder 200 to spill out of the powder feeder 70. In the powder feeder 70 according to the third configuration example in FIG. 10, the powder 200 is prevented from spilling out by setting the gas inlet OP1 smaller than that in the first configuration example, instead of providing the mesh 77. As in the powder feeder 70 according to the first configuration example, the powder 200 is diffused by the airflow supplied from the tube 13.

The air supply portion 73 and the discharge portion 76 may be provided with covers for preventing leakage of the powder or moisture ingress. These covers are removed when, for example, the powder feeder 70 is attached to the distal end portion 7 or the insertion portion 5 of the endoscope main body 1A to which the powder feeder 70 is attached is inserted into the living body 100.

FIG. 11 illustrates a fourth configuration example of the powder feeder 70. The powder feeder 70 according to the fourth configuration example in FIG. 11 is different from the powder feeder 70 according to the first configuration example in FIG. 8 in the arrangement position and shape of the discharge portion 76, the presence or absence of the mesh 77, the size of the gas inlet OP1, and the presence or absence of a mesh 79. The discharge portion 76 in the fourth configuration example has a bottomed cylindrical shape. A bottom BT1 of the discharge portion 76 is disposed at a position where the airflow flowing into a powder accommodation space SP4 from the gas inlet OP1 hits the bottom BT1. The airflow that has hit the bottom BT1 is turned into an air turbulence and functions to diffuse the powder 200. The discharge portion 76 has a plurality of powdery fluid outlets EXI provided on a side surface of the bottomed cylindrical discharge portion 76. The reason why the gas inlet OP1 in the fourth configuration example is smaller is the same as that in the third configuration example.

In the powder feeders 70 illustrated in FIGS. 8 to 11, the powder accommodation spaces SP1, SP2, SP3, and SP4 are provided outside the tube 13. However, a part of the powder accommodation spaces SP1, SP2, SP3, and SP4 may be provided inside the tube 13.

In addition, the direction of the powdery fluid discharged from the discharge portion 76 may not be parallel to the flow direction of the gas in the tube 13. For example, the powder feeder 70 may be configured such that the direction of the powdery fluid discharged from the discharge portion 76 and the flow direction of the gas in the tube 13 intersect at a predetermined angle.

The first embodiment has described the case where the accommodation portion 74 and the diffusion portion 75 are provided in the same chamber, but the accommodation portion 74 and the diffusion portion 75 may be provided in different chambers. It is to be noted, however, that it is preferable to provide the accommodation portion 74 and the diffusion portion 75 in the same chamber, because the place where the powder 200 is left is reduced.

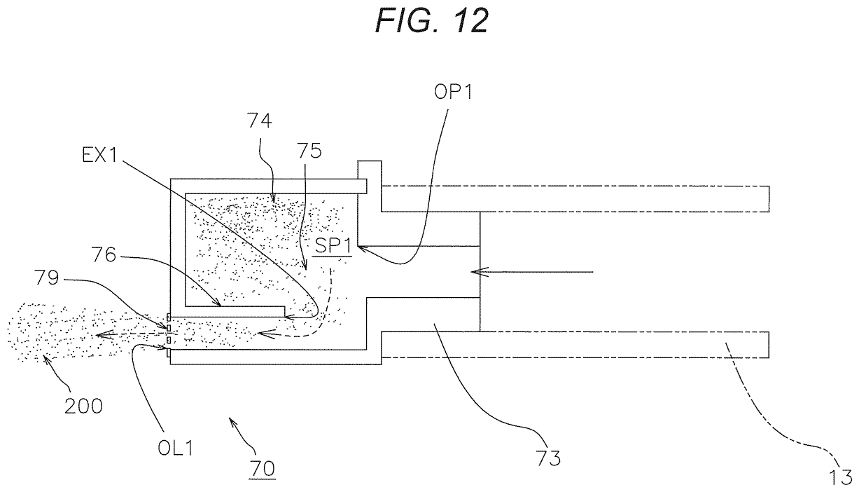

The first embodiment has described the example in which the mesh is not provided in the discharge port OL1, but the mesh may be provided in the discharge port OL1. A powder feeder 70 according to the fifth configuration example illustrated in FIG. 12 has a configuration in which a mesh 79 is provided at the discharge port OL1 of the powder feeder 70 according to the first configuration example illustrated in FIG. 8. Note that, in the powder feeder 70 according to the fifth configuration example, the mesh 77 provided in the gas inlet OP1 of the powder feeder 70 according to the first configuration example is eliminated. Meanwhile, in the powder feeder 70 according to the fifth configuration example illustrated in FIG. 12, the position of the discharge portion 76 and the positions of the accommodation portion 74 and the diffusion portion 75 are vertically reversed as compared with the powder feeder 70 in FIG. 8. That is, the discharge portion 76 in FIG. 8 is positioned on the upper side, whereas the discharge portion 76 in FIG. 12 is positioned on the lower side. For example, when the powder feeder 70 according to the fifth configuration example was subjected to a powder feed experiment by actually switching the position of the discharge portion 76 in the vertical direction, there was no large difference in the discharge of the powder 200, and the powder 200 was satisfactorily diffused and supplied in any case.

### Second Embodiment

### (4) Powder Feeder According to Second Embodiment

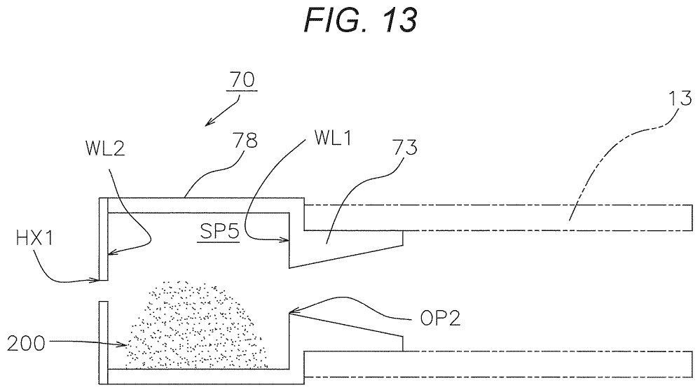

A powder feeder 70 according to the second embodiment includes an air supply portion 73 and an accommodation case 78 that serves as an accommodation portion, a diffusion portion, and a discharge portion as illustrated in FIG. 13. The accommodation case 78 in the second embodiment is a case made of resin, for example. The air supply portion 73 functions as a fitting portion to be fitted into a tube 13 which is an air supply nozzle. The accommodation case 78 is disposed in a region AR indicated by a dash-dotted line in FIG. 7 when a distal end portion 7 is viewed in the insertion direction of the distal end portion 7. The accommodation case 78 is fixed to a main body 71 of the powder feeder 70.

The accommodation case 78 serving as the accommodation portion, the diffusion portion, and the discharge portion functions as a container for accommodating powder 200 therein. A gas inlet OP2 communicating with the air supply portion 73 is provided in a first wall surface WL1 of the accommodation case 78. A discharge hole HX1 as a powdery fluid outlet is provided in a second wall surface WL2 different from the first wall surface WL1 of the accommodation case 78.

The accommodation case 78 also functions as a diffusion portion that diffuses the powder 200 into a gas by the gas entering through the gas inlet OP2. For this reason, the volume of a powder accommodation space SP5 of the accommodation case 78 is preferably 1.1 times or more the volume of the powder 200 that is accommodated. The powder accommodation space SP5 that is sufficiently wide as described above can diffuse the powder 200 into the gas supplied through the air supply portion 73. The accommodation case 78 also has a function of a discharge portion that discharges, from the discharge hole HX1, a powdery fluid obtained by diffusing the powder 200 into the gas.

In order to diffuse the powder 200 in the powder accommodation space SP5, the opening areas of the gas inlet OP2 and the discharge hole HX1 are preferably smaller than the cross-sectional area of the tube 13.

In the powder feeder 70 illustrated in FIG. 13, the gas inlet OP2 and the discharge hole HX1 are disposed on the extension of the flow of gas in the tube 13. In other words, the outlet of the tube 13, the gas inlet OP2, and the discharge hole HX1 are linearly arranged. However, the gas inlet OP2 and the discharge hole HX1 may not be arranged on the extension of the flow of the gas in the tube 13. For example, the gas inlet OP2 and the discharge hole HX1 may be arranged such that the direction of the gas passing through the gas inlet OP2 and/or the discharge hole HX1 intersects with the flow of the gas in the tube 13.

In the powder feeder 70 illustrated in FIG. 13, the powder accommodation space SP5 is provided outside the tube 13. However, a part of the powder accommodation space SP5 may be provided inside the tube 13.

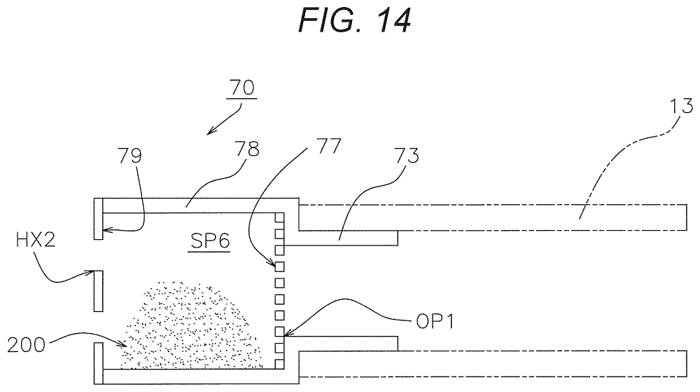

The second embodiment has described the example in which a mesh is not provided in the discharge hole HX1, but the mesh may be provided in the discharge hole HX1. Assuming that the accommodation case 78 has a cylindrical shape, the configuration approaches the configuration of a powder feeder 70 according to a third embodiment to be described later illustrated in FIG. 14 by providing a mesh with the size of the discharge hole HX1 coinciding with the inner diameter of the accommodation case 78.

### Third Embodiment

### (5) Powder Feeder According to Third Embodiment

The powder feeder 70 according to the third embodiment includes an air supply portion 73 and an accommodation case 78 that serves as an accommodation portion, a diffusion portion, and a discharge portion as illustrated in FIG. 14. The air supply portion 73 functions as a fitting portion to be fitted into a tube 13 which is an air supply nozzle. The accommodation case 78 is disposed in a region AR indicated by a dash-dotted line in FIG. 7 when a distal end portion 7 is viewed in the insertion direction of the distal end portion 7. The accommodation case 78 is fixed to a main body 71 of the powder feeder 70. The accommodation case 78 in the third embodiment is a case made of resin, for example.

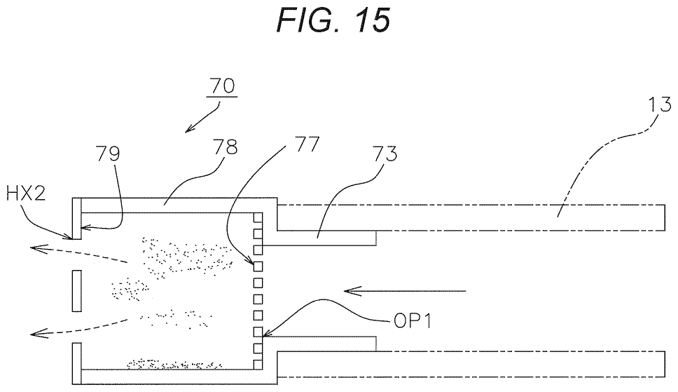

The accommodation case 78 serving as the accommodation portion, the diffusion portion, and the discharge portion functions as a container for accommodating powder 200 therein. The powder 200 is accommodated in a powder accommodation space SP6 of the accommodation case 78. A gas inlet OP1 communicating with the air supply portion 73 is provided with a mesh 77. Mesh openings of a mesh 79 serve as discharge holes HX2 of the accommodation case 78. Although the meshes 77 and 79 in FIGS. 14 and 15 are schematically illustrated for easy viewing, they have a large number of mesh openings.

The mesh 77 disposed at the gas inlet OP1 is for preventing a backflow of the powder 200, that is, for preventing the powder 200 from flowing into the tube 13 from the powder accommodation space SP6. From the mesh 79 for forming the plurality of discharge holes HX2, a powdery fluid that contains the powder 200 diffused in the gas supplied from the air supply portion 73 is ejected. Due to the difference in function as described above, the mesh openings of the mesh 79 for forming the discharge holes HX2 are larger than the mesh openings of the mesh 77 arranged at the gas inlet OP1.

The accommodation case 78 also functions as a diffusion portion that diffuses the powder 200 into the gas by the gas entering through the gas inlet OP1. For this reason, the volume of the powder accommodation space SP6 of the accommodation case 78 is preferably 1.1 times or more the volume of the powder 200 that is accommodated. The powder accommodation space SP6 that is sufficiently wide as described above can contribute to diffusing the powder 200 into the gas supplied through the air supply portion 73. The accommodation case 78 also functions as a discharge portion for discharging the powdery fluid obtained by diffusing the powder 200 into a gas through a plurality of discharge holes HX2 (mesh openings of the mesh 79). FIG. 15 schematically illustrates a state in which the powder 200 is diffused in the accommodation case 78 and discharged as the powdery fluid through the mesh 79.

In the powder feeder 70 illustrated in FIG. 14, the meshes 77 and 79 are disposed on the extension of the flow of gas in the tube 13. In other words, the outlet of the tube 13, the mesh 77, and the mesh 79 are linearly arranged. However, the meshes 77 and 79 may not be provided on the extension of the flow of gas in the tube 13. For example, the mesh 77 and the mesh 79 may be arranged such that the direction of the gas passing through the mesh 77 and/or the mesh 79 intersects with the flow of the gas in the tube 13.

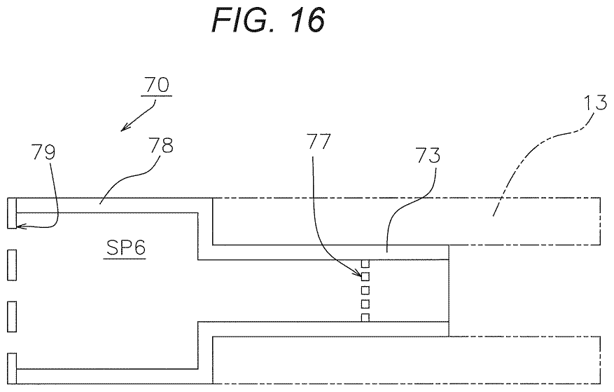

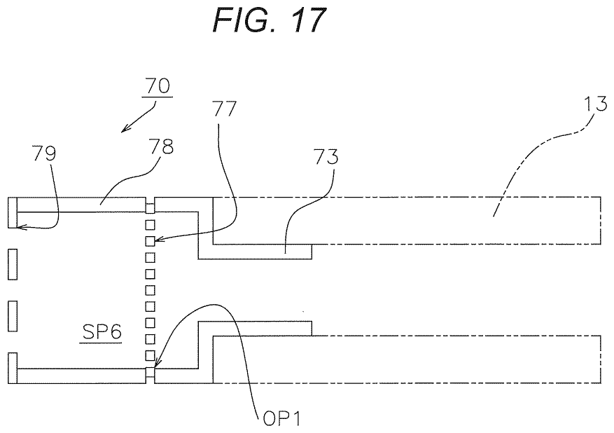

In the powder feeder 70 illustrated in FIG. 14, the powder accommodation space SP6 is provided outside the tube 13. However, as illustrated in FIG. 16, a part of the powder accommodation space SP6 may be provided in the tube 13 by arranging the mesh 77 in the tube 13. Conversely, as illustrated in FIG. 17, the mesh 77 may be provided at a position away from the tube 13 so as to make the flow path area on the upstream side of the mesh 77 same as the flow path area on the downstream side of the mesh 77.

### Fourth Embodiment

### (6) Powder Feeder According to Fourth Embodiment

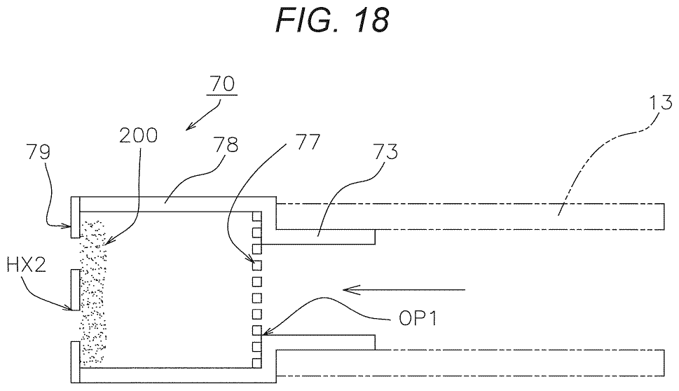

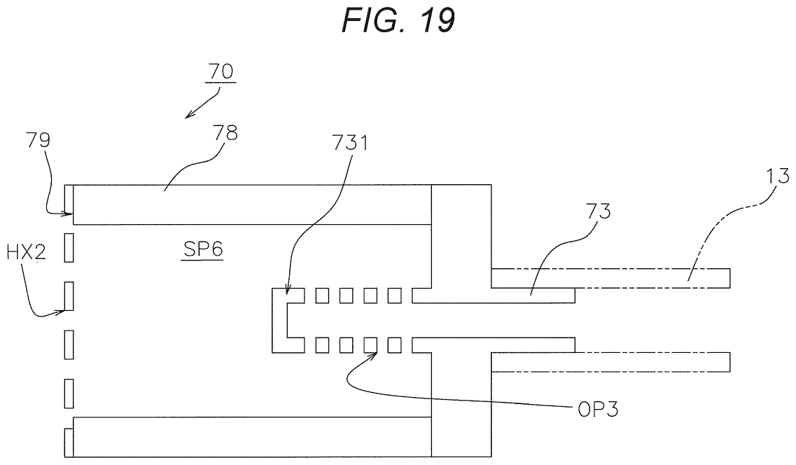

In the powder feeder 70 according to the third embodiment described above, there is a possibility of clogging that blocks the discharge holes HX2 of the mesh 79 included in the discharge portion of the accommodation case 78 as illustrated in FIG. 18. In view of this, a powder feeder 70 according to the fourth embodiment illustrated in FIG. 19 is obtained by improving the method for ejecting a gas from the air supply portion 73 to the accommodation case 78 as compared with the powder feeder 70 according to the third embodiment. The powder feeder 70 according to the fourth embodiment includes a supply nozzle 731 protruding into a powder accommodation space SP6. A gas inlet OP3 is formed in the supply nozzle 731. The flow of a gas entering the powder accommodation space SP6 through the gas inlet OP3 is not parallel to the flow of the gas in the tube 13. In other words, the flow of the gas entering the powder accommodation space SP6 through the gas inlet OP3 intersects with the flow of the gas in the tube 13. The discharge hole HX2 is oriented in a direction orthogonal to the direction of the opening of the tube 13. Note that, although the case where the direction of the discharge hole HX2 is not parallel to the direction of the opening of the tube 13 has been described here, the direction of the discharge hole HX2 and the direction of the opening of the tube 13 may be parallel to each other, and the gas discharged through the discharge hole HX2 does not directly hit the mesh 79 (hits the wall of the accommodation case 78). Even with such a configuration, clogging of the mesh 79 with the powder 200 can be reduced.

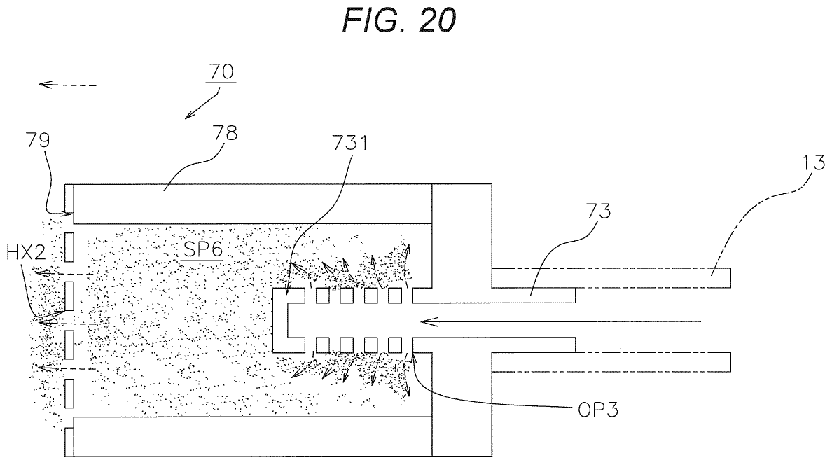

As illustrated in FIG. 20, the powder feeder 70 according to the fourth embodiment can promote the diffusion of the powder in the powdery fluid that has reached the vicinity of the mesh 79 (because the number of powder lumps is small) as compared with the powder feeder 70 according to the third embodiment, and can reduce the possibility of causing clogging of the mesh 79.

### Fifth Embodiment

### (7) Powder Feeder According to Fifth Embodiment

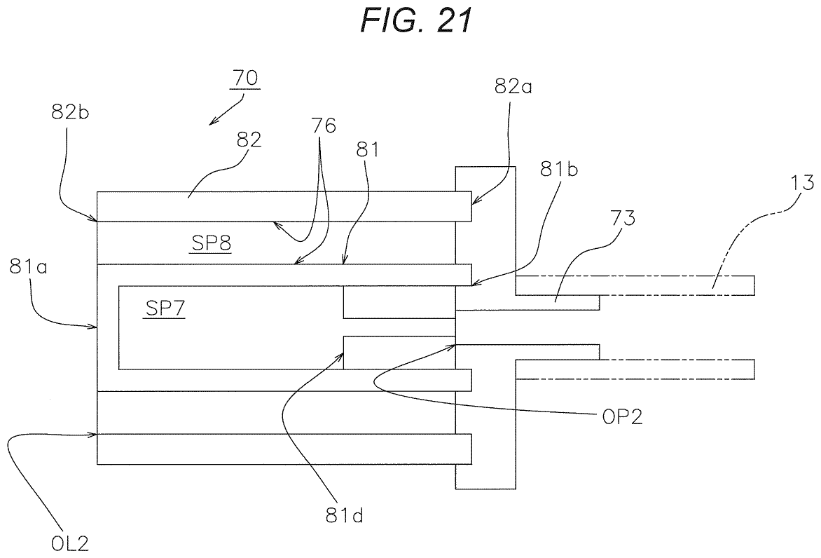

A powder feeder 70 according to the fifth embodiment illustrated in FIG. 21 is different from the powder feeder 70 according to the first embodiment illustrated in FIG. 8 in that a discharge hole OL2 has an annular shape. An inner cylinder 81 having a bottomed cylindrical shape and an outer cylinder 82 having both ends opened are attached to an air supply portion 73. The inner cylinder 81 and the outer cylinder 82 are disposed such that, for example, the central axes thereof coincide with each other.

The space in the inner cylinder 81 serves as a powder accommodation space SP7. A region forming the space SP8 between the inner cylinder 81 and the outer cylinder 82 serves as a discharge portion 76. The powder accommodation space SP7 and the space SP8 of the discharge portion 76 are in communication with each other through a notch 81d described later. The notch 81d is a powdery fluid outlet of the first chamber and also a powdery fluid inlet of a second chamber.

The inner cylinder 81 has a bottom 81a at one end, an opening 81b at the other end, and a cylindrical side surface 81c between the bottom 81a and the opening 81b. Four notches 81d extending from the opening 81b toward the bottom 81a are formed in the side surface 81c. The length of each notch 81d in the axial direction is, for example, 2 mm, and the width is, for example, 2 mm. The four notches 81d are disposed so as to overlap each other, for example, when the inner cylinder 81 is rotated by 90 degrees about the central axis. An outer diameter of the outer cylinder 82 is, for example, 12 mm, and a gap between the side surface 81c of the inner cylinder 81 and the outer cylinder 82 is, for example, 2 mm.

The opening 81b is in communication with the gas inlet OP2 of the air supply portion 73. A section outside the region of the opening 81b communicating with the gas inlet OP2 is closed by the air supply portion 73. Therefore, the gas entering through the gas inlet OP2 enters the space SP8 of the discharge portion 76 through the notches 81d. One end 82a of the outer cylinder 82 is attached to the air supply portion 73. Therefore, the one end 82a of the outer cylinder 82 is closed by the air supply portion 73 and the inner cylinder 81. The bottom 81a of the inner cylinder 81 is located at the other end 82b of the outer cylinder 82. A section of the opening at the other end 82b of the outer cylinder 82 that is not closed by the bottom 81a is the annular discharge hole OL2.

The gas entering through the gas inlet OP2 of the air supply portion 73 diffuses the powder accommodated in the powder accommodation space SP7 to generate a powdery fluid, and enters the space SP8 of the discharge portion 76 through the notches 81d. The powdery fluid having entered the discharge portion 76 is discharged from the annular discharge hole OL2. As described above, the inner cylinder 81 having the powder accommodation space SP7 also functions as an accommodation portion and a diffusion portion. The gas flowing parallel to the central axis of the inner cylinder 81 toward the gas inlet OP2 rapidly changes its direction to pass through the notches 81d. Such a gas flow causes diffusion of the powder accommodated in the powder accommodation space SP7.

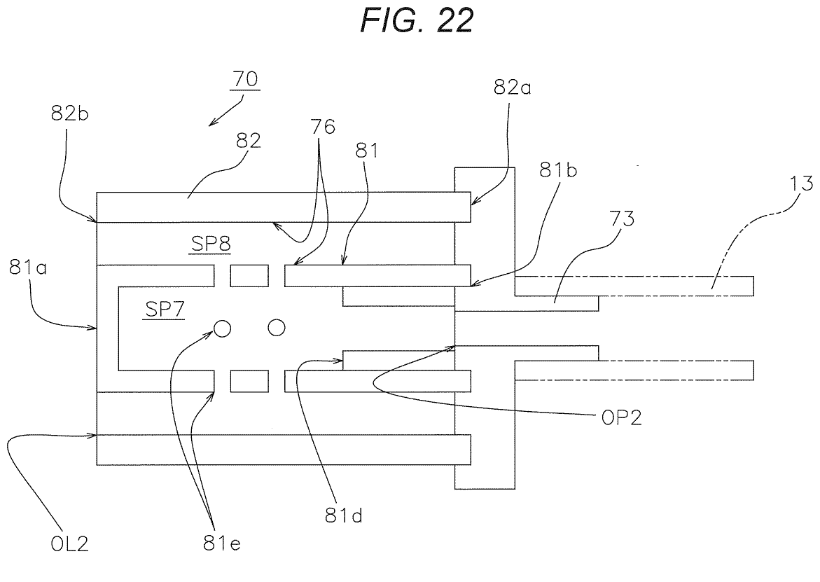

FIG. 22 illustrates another example of the powder feeder 70 according to the fifth embodiment. The powder feeder 70 in FIG. 22 is different from the powder feeder 70 in FIG. 21 in that eight small holes 81e, for example, are provided. The eight small holes 81e are arranged to form four rows of two small holes 81e in the central axis direction. By providing the plurality of small holes 81e in the side surface 81c of the inner cylinder 81 as described above, the gas smoothly flows from the powder accommodation space SP7 of the inner cylinder 81 into the space SP8 of the discharge portion 76. The hole diameter of the small hole 81e is, for example, 1 mm. The number and arrangement of the small holes 81e illustrated in FIG. 22 are an example, and the number and arrangement of the small holes 81e can be changed.

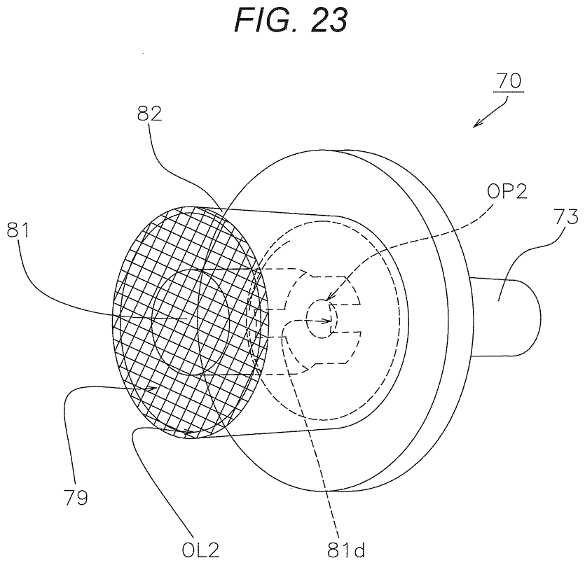

FIG. 23 illustrates another example of the powder feeder 70 according to the fifth embodiment. The powder feeder 70 in FIG. 23 is different from the powder feeder 70 in FIG. 21 in that a mesh 79 is provided at the discharge hole OL2. The attachment of the mesh 79 to the discharge hole OL2 can prevent the powder from flying out at once, and the diffusion of the powder is promoted. Similarly, a mesh may be provided in the air supply portion 73 to prevent the powder from spilling through the gas inlet OP2.

### Sixth Embodiment

### (8) Powder Feeder According to Sixth Embodiment

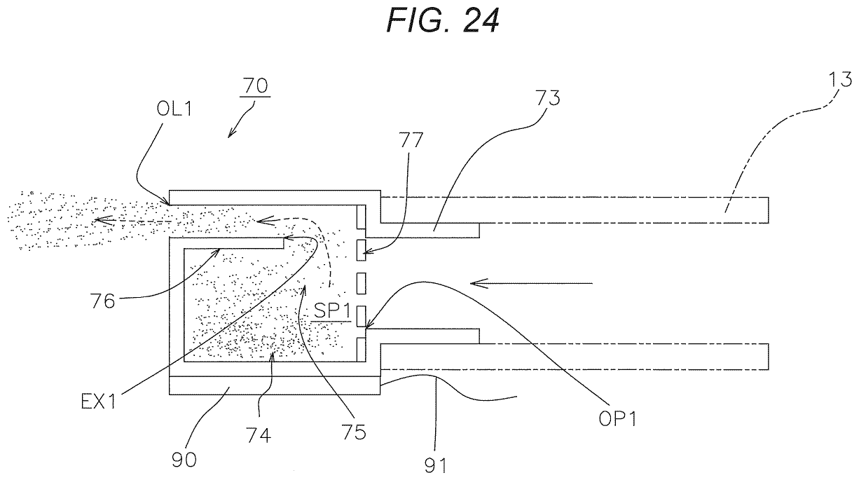

A powder feeder 70 according to the sixth embodiment illustrated in FIG. 24 is different from the powder feeder 70 according to the first embodiment illustrated in FIG. 8 in further having a vibrating device 90. The vibrating device 90 is a device that vibrates the powder accommodated in an accommodation portion 74. In the sixth embodiment, the vibrating device 90 can be operated from the outside of an endoscope main body 1A.

The vibrating device 90 can be composed of, for example, a piezoelectric vibrator. The vibrating device 90 can be obtained by attaching a piezoelectric vibrator to the powder feeder 70 and connecting an AC power supply to the piezoelectric vibrator from the outside. A power supply line 91 that supplies power to the vibrating device 90 can be wired through a forceps channel 9, for example.

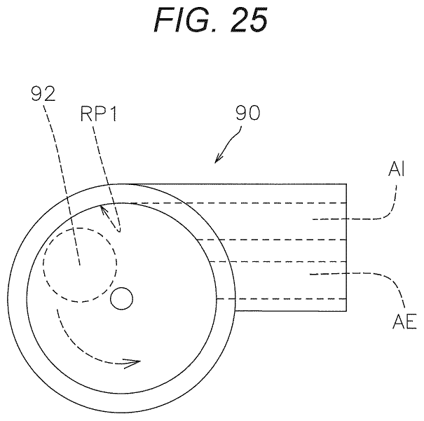

The vibrating device 90 may alternatively be a device that generates vibration by an airflow illustrated in FIG. 25. The vibrating device 90 in FIG. 25 is configured such that a sphere 92 can move in an annular path RP1. An air inlet AI and an air outlet AE communicate with the annular path RP1. Air is sucked through the air inlet AI and discharged through the air outlet AE, by which a flow of air circulating through the annular path RP1 is generated. The sphere 92 circulates in the annular path RP1 by the flow of air in the annular path RP1, whereby the vibrating device 90 vibrates. A dashed arrow in FIG. 25 indicates the rotation direction of the sphere 92.

The powder feeder 70 according to the sixth embodiment vibrates the accommodation portion 74 by the vibrating device 90 to vibrate the powder when a gas is supplied from the tube 13. The powder feeder vibrates the powder in this manner, whereby the powder feeder 70 can easily diffuse the powder 200 in the powder accommodation space SP1. Since the diffusion is facilitated, the aggregation of the powder and adhesion of the powder to the side surface of the accommodation portion 74 can be suppressed, and uneven distribution of the powder 200 in the gas is reduced.

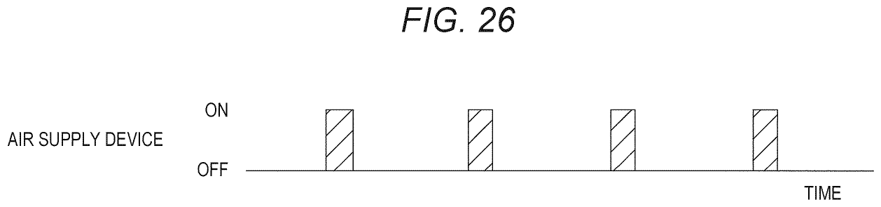

FIG. 26 illustrates an example of a method for supplying air using an air supply device 30. The air supply device 30 is not turned on throughout an air supply period, but is repeatedly turned on and off during the air supply period. When the air supply device 30 is operated in this manner, the supplied air pulsates, so that the pressure of the gas sent to the air supply portion 73 repeatedly changes. The air supply device 30 can apply vibration to the powder 200 in the powder feeder 70 due to a change in pressure of the gas when the powder 200 is discharged from the powder feeder 70. The powder 200 is diffused by the airflow while being vibrated so that the powder 200 can be easily diffused.

The method for applying vibration to the powder 200 by the air supply device 30 is not limited to the method for turning on and off the air supply device 30. For example, the air supply device 30 may change the pressure of the supplied air while continuously feeding the gas, or may supply air so that sound waves are transmitted in the gas.

### Seventh Embodiment

### (9) Powder Feeder According to Seventh Embodiment

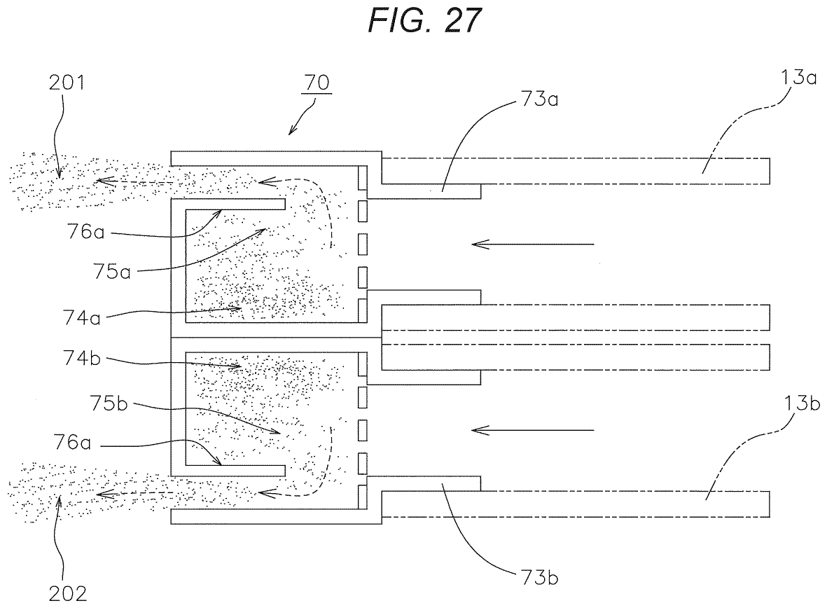

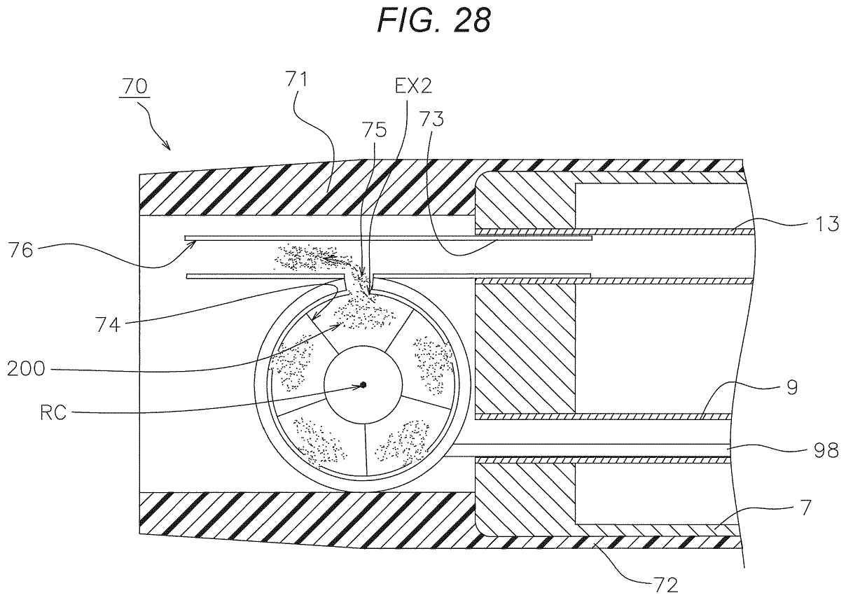

As illustrated in FIG. 27, a powder feeder 70 according to the seventh embodiment includes a first air supply portion 73a, a second air supply portion 73b, a first accommodation portion 74a, a second accommodation portion 74b, a first diffusion portion 75a, a second diffusion portion 75b, a first discharge portion 76a, and a second discharge portion 76b. Two tubes 13 are attached to the powder feeder 70.

The configuration including the first air supply portion 73a, the first accommodation portion 74a, the first diffusion portion 75a, and the first discharge portion 76a is similar to that of the powder feeder 70 according to the first embodiment. The configuration including the second air supply portion 73b, the second accommodation portion 74b, the second diffusion portion 75b, and the second discharge portion 76b is similar to that of the powder feeder 70 according to the first embodiment. In short, the powder feeder 70 according to the seventh embodiment is obtained by combining the two powder feeders 70 according to the first embodiment. Since the configuration of each portion of the powder feeder 70 has already been described in the first embodiment, the description thereof will be omitted in the seventh embodiment.

The powder feeder 70 according to the seventh embodiment includes, as the accommodation portion, the first accommodation portion 74a that accommodates a first powder 201 and the second accommodation portion 74b that accommodates a second powder 202. The powder feeder 70 according to the seventh embodiment includes the first air supply portion 73a and the second air supply portion 73b as air supply portions. Therefore, the first air supply portion 73a and the second air supply portion 73b can individually and independently supply a gas to the first accommodation portion 74a and the second accommodation portion 74b.

The powder feeder 70 according to the seventh embodiment also includes the first diffusion portion 75a, the second diffusion portion 75b, the first discharge portion 76a, and the second discharge portion 76b as diffusion portions and discharge portions. The powder feeder 70 according to the seventh embodiment is configured to, when a gas is supplied to the first accommodation portion 74a, diffuse the first powder 201 into the gas in the first diffusion portion 75a to generate a first powdery fluid, and discharge the first powdery fluid from the first discharge portion 76a. The powder feeder 70 according to the seventh embodiment is configured to, when a gas is supplied to the second accommodation portion 74b, diffuse the second powder 202 into the gas in the second diffusion portion 75b to generate a second powdery fluid, and discharge the second powdery fluid from the second discharge portion 76b.

In the powder feeder 70 according to the seventh embodiment, when, for example, the first powder 201 and the second powder 202 have the same composition, the second powder 202 can be used as a reserve for the first powder 201, and the second powder 202 can be used when the treatment is not completed only with the first powder 201. In addition, if the first powder 201 and the second powder 202 have different compositions, a treatment that needs to be performed with two different types of powders can be completed by one insertion of the powder feeder 70 without replacing the powder feeder 70. Furthermore, even when there are two sites to be treated, the treatment can be completed by one insertion without replacing the powder feeder 70.

In the above description, the case where the powder feeder 70 according to the seventh embodiment has a configuration of combining two powder feeders 70 having the configuration illustrated in FIG. 8 has been described. However, another configuration may be combined. For example, the configurations illustrated in FIGS. 9, 10, 11, 12, 13, 14, 15, 16, or 17 may be combined. In addition, the configurations to be combined may not be the same, and for example, the configuration illustrated in FIG. 8 and the configuration illustrated in FIG. 14 may be combined.

### Eighth Embodiment

### (10) Powder Feeder According to Eighth Embodiment

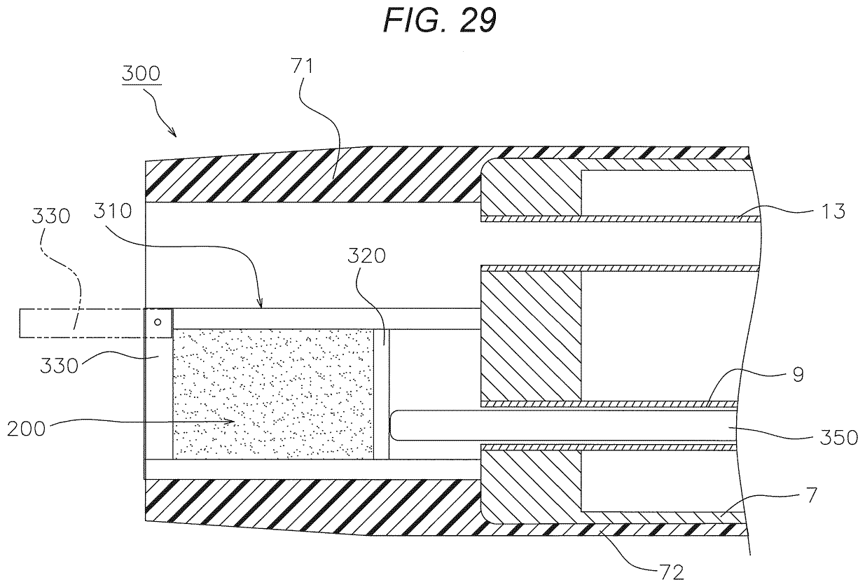

A powder feeder 70 according to the eighth embodiment includes an air supply portion 73, five accommodation portions 74, a diffusion portion 75, a discharge portion 76, and a string 98 for moving the accommodation portions 74 as illustrated in FIG. 28. The five accommodation portions 74 are configured to be rotatable about an axis RC. Each of the accommodation portions 74 accommodates powder 200. Each of the accommodation portions 74 has a powdery fluid outlet EX2. When the powdery fluid outlet EX2 of each of the accommodation portions 74 coincides with the diffusion portion 75, the powdery fluid containing the powder 200 flows out from the diffusion portion 75 to the discharge portion 76. The pressure in the discharge portion 76 is lower than that in the accommodation portion 74 due to the fast flow of the gas from the air supply portion 73 toward the discharge portion 76, so that the powder 200 is sucked out toward the discharge portion 76.

The string 98 is wound around the accommodation portion 74 (not illustrated). The string 98 is repeatedly pulled by a predetermined distance, whereby the powdery fluid outlet EX2 of each accommodation portion 74 sequentially coincides with the diffusion portion 75. As described above, the powdery fluid outlet EX2 sequentially coincides with the diffusion portion 75, whereby the powder 200 accommodated in each accommodation portion 74 is sequentially discharged.

The powder feeder 70 according to the eighth embodiment illustrated in FIG. 28 has a plurality of accommodation portions 74, and can switch the accommodation portion 74 accommodating the powder 200 to be fed to the diffusion portion 75 by a power source. Here, the string 98 has been described as an example of the power source, but the power source may be, for example, a rod, a gas, or a liquid.

### Others

### (11) Other Powder Feeders

FIG. 29 illustrates an example of another powder feeder 300 that feeds, unlike the embodiments, powder without diffusing the powder into a gas. The powder feeder 300 includes a case 310, a spacer 320, a lid 330, and a rod 350. The powder 200 is accommodated in the case 310. The spacer 320 is configured to be movable by being pushed by the rod 350. When the spacer 320 is pushed and moved by a wire, the powder 200 is pushed by the spacer 320. When the powder 200 is pushed by the spacer 320, the lid 330 is opened by being pushed by the powder 200. When the spacer 320 is continuously pushed after the lid 330 is opened, the powder 200 is supplied into a living body 100 from the lid that is opened. It is to be noted that, in the powder feeder 300, the powder 200 is supplied so as to fall down in the form of powder instead of the powdery fluid. Here, the case where the rod 350 is used to push the spacer 320 has been described, but the spacer 320 may be pushed by air or water supplied using the tube 13 instead of the rod 350.

Here, the configuration in which the powder 200 is pushed out by the rod 350 has been described, but the powder feeder 300 may be changed in orientation, for example, may be turned upside down, by the rod 350 to allow the powder 200 to fall down from the powder feeder 300. As compared with the powder feeder 300, the powder feeders 70 according to the above embodiments are excellent in that powder can be evenly dispersed to a specific part.

### (12) Features

(12-1)

The powder feeder 70 according to each of the first to eighth embodiments is attached to the distal end portion 7 of the insertion portion 5 of the endoscope main body 1A to be inserted into the living body 100. The powder feeder 70 is a device that feeds the powder 200 into the living body 100. The powder feeder 70 attached to the distal end portion 7 of the insertion portion 5 by the attachment portion 72 takes in a gas from the tube 13 which is an air supply nozzle of the endoscope main body 1A by the air supply portion 73, and accommodates the powder 200 in the accommodation portion 74. The powder feeder 70 diffuses the powder 200 into the gas by the diffusion portion 75, and discharges the powdery fluid (gas and powder) into which the powder 200 has been diffused into the living body 100 by the discharge portion 76.

The air supply portion 73 communicates with the accommodation portion 74, the accommodation portion 74 communicates with the diffusion portion 75, and the diffusion portion 75 communicates with the discharge portion 76. Therefore, the gas supplied from the tube 13 to the air supply portion 73 is blown into the living body 100 through the air supply portion 73, the accommodation portion 74, the diffusion portion 75, and the discharge portion 76.

The powder is diffused into the gas in the powder feeder 70, in other words, in the living body 100. Therefore, as compared with the case where the powder and the gas are supplied together from the outside of the living body 100, the amount of the powder 200 required is small even if the amount of the powder 200 reaching the inside of the living body 100 is the same. For example, by using the powder feeder 70, the amount of the powder 200 to be used can be reduced by the amount of the powder 200 that is left in the tube 13. In addition, the use of the powder feeder 70 can prevent a situation in which sufficient powder 200 cannot be sent into the living body 100 due to the tube 13 being clogged with the powder 200. That is, by using the powder feeder 70, it is possible to sufficiently feed powder to a specific part in the living body 100 and to reduce the loss of powder.

As described in the above embodiments, some functions of the air supply portion 73, the accommodation portion 74, the diffusion portion 75, and the discharge portion 76 can be shared by one section. For example, the accommodation case 78 serves as an accommodation portion, a diffusion portion, and a discharge portion. The air supply portion 73 and the accommodation portion 74 can be implemented by one section. The accommodation portion 74 and the diffusion portion 75 can be implemented by one section. The diffusion portion 75 and the discharge portion 76 can be implemented by one section. The air supply portion 73, the accommodation portion 74, and the diffusion portion 75 can be implemented by one section.

(12-2)

In the powder feeder 70 according to each of the first to eighth embodiments, the air supply portion 73, the accommodation portion 74, the diffusion portion 75, and the discharge portion 76 are disposed at positions where the objective lens 11 and the light guide lens 12 at the distal end portion 7 are exposed in a state where the attachment portion 72 is attached to the distal end portion 7. With the exposed objective lens 11 and light guide lens 12, visual information can be obtained by the endoscope 1 to which the powder feeder 70 is attached.

(12-3)

In the powder feeder 70 according to each of the third embodiment and the fourth embodiment, the air supply portion 73 serving as a fitting portion is inserted into the tube 13, and the accommodation case 78 and the mesh 79 function as an accommodation portion, a diffusion portion, and a discharge portion. The accommodation case 78 and the mesh 79 diffuse the powder 200 accommodated therein by the gas into the gas, and discharge the powdery fluid from an opening.

In each of the third embodiment and the fourth embodiment, the powder feeder 70 capable of sufficiently supplying powder to a specific part and reducing the loss of powder can be achieved with a simple configuration obtained by adding the air supply portion 73, the accommodation case 78, and the mesh 79 to the main body 71 and the attachment portion 72.

(12-4)

In the powder feeder 70 according to the second embodiment, the air supply portion 73 serving as a fitting portion is inserted into the tube 13, and the accommodation case 78 functions as an accommodation portion, a diffusion portion, and a discharge portion. The accommodation case 78 includes a gas inlet OP2 formed in the first wall surface WL1 and a discharge hole HX1 formed in the second wall surface WL2 as a powdery fluid outlet. The accommodation case 78 diffuses the powder 200 into the gas with the gas entering through the gas inlet OP2 and discharges the powdery fluid through the discharge hole HX1.

In the second embodiment, the powder feeder 70 capable of sufficiently supplying powder to a specific part and reducing the loss of powder can be achieved with a simple configuration obtained by adding the air supply portion 73 and the accommodation case 78 to the main body 71 and the attachment portion 72.

(12-5)

The powder feeder 70 according to the first embodiment includes a first chamber functioning as the accommodation portion 74 and the diffusion portion 75 and a second chamber functioning as the discharge portion 76. The first chamber includes the gas inlet OP1 communicating with the air supply portion 73, the powdery fluid outlet EX1 communicating with the discharge portion 76, and any of the powder accommodation spaces SP1 to SP4, diffuses the powder 200 into the gas by the gas blown through the gas inlet OP1, and discharges the powdery fluid through the powdery fluid outlet EX1. The discharge portion 76 has a discharge port OL1 facing the inside of the living body 100 and a powdery fluid inlet also serving as the powdery fluid outlet EX1, and has a volume smaller than that of any of the powder accommodation spaces SP1 to SP4.

In the first embodiment, the powder feeder 70 capable of sufficiently supplying powder to a specific part and reducing the loss of powder can be achieved with a simple configuration obtained by adding, to the main body 71 and the attachment portion 72, the first chamber functioning as the accommodation portion 74 and the diffusion portion 75 and the second chamber functioning as the discharge portion 76.

(12-6)

The vibrating device 90 illustrated in FIG. 24 vibrates the powder 200 accommodated in the accommodation portion 74. The powder 200 can be vibrated when the powder 200 is diffused into the gas, so that aggregation of the powder and adhesion of the powder to the side surface of the accommodation portion 74 can be prevented as compared with a case where the powder is not vibrated. Thus, the powder 200 can be diffused so as to reduce uneven distribution in the gas.

(12-7)

The powder feeder 70 illustrated in FIG. 27 discharges the first powdery fluid obtained by diffusing the first powder 201 into the gas by supplying the gas to the first accommodation portion 74a. The powder feeder 70 illustrated in FIG. 27 also discharges the second powdery fluid obtained by diffusing the second powder 202 into the gas by supplying the gas to the second accommodation portion 74b. The powder feeder 70 in FIG. 27 can discharge a plurality of types of powder by using the first powder 201 and the second powder 202 which are different from each other. In addition, the powder feeder 70 in FIG. 27 can discharge the first powder 201 and the second powder 202 at different timings.

(12-8)

As illustrated in FIG. 26, the air supply device 30 can apply vibration to the powder 200 in the powder feeder 70 due to a change in pressure of the gas when the powder 200 is discharged from the powder feeder 70. As a result, the powder 200 to be discharged from the powder feeder 70 can be easily diffused.

(12-9)

A method for using the powder feeder 70 includes a replacement step, a reinsertion step, and a powdery fluid discharge step. In the replacement step, after the insertion portion 5 of the endoscope 1 is inserted into the living body 100 to treat a specific part in the living body 100, the insertion portion 5 is pulled out from the living body 100, and the powder feeder 70 that accommodates the powder 200 is replaced with the hood 20 at the distal end portion 7 of the insertion portion 5 and is connected to the tube 13 for air supply. In the maximum insertion step, the powder feeder 70 is inserted into the living body with the insertion portion 5 to which the powder feeder 70 is attached being directed to the specific part in the living body 100. In the powdery fluid discharge step, a gas is fed to the powder feeder 70 located in the living body 100, and the powdery fluid containing powder is discharged from the powder feeder 70 to the specific part.

According to such a method for using the powder feeder 70, the powder 200 can be delivered to the vicinity of the specific part in the state of being accommodated in the powder feeder 70, and the powdery fluid is generated by the diffusion portion 75 in the living body 100, so that the loss of the powder 200 can be reduced when the powder 200 is sufficiently supplied to the specific part in the living body 100.

Although embodiments of the present disclosure have been described above, the present invention is not limited to the above embodiments, and various modifications can be made without departing from the gist of the invention. In particular, the plurality of embodiments and modifications described in the present specification can be combined in any manner as necessary.

1. A powder feeder that is to be attached to a distal end portion of an insertion portion of an endoscope main body to be inserted into a living body and that supplies powder into the living body, the powder feeder comprising:
an attachment portion configured to be attached to the distal end portion of the insertion portion;
an air supply portion configured to take in a gas from an air supply nozzle of the endoscope main body;
an accommodation portion communicating with the air supply portion and configured to accommodate the powder;
a diffusion portion communicating with the accommodation portion and configured to diffuse the powder accommodated in the accommodation portion into the gas fed from the air supply nozzle; and
a discharge portion communicating with the diffusion portion and configured to discharge the gas into which the powder has been diffused into the living body together with the powder.

2. The powder feeder according to claim 1, wherein
the air supply portion, the accommodation portion, the diffusion portion, and the discharge portion are disposed at positions where an objective lens and a light guide are exposed at the distal end portion in a state where the attachment portion is attached to the distal end portion.

3. The powder feeder according to claim 1, wherein
the accommodation portion, the diffusion portion, and the discharge portion are defined by an accommodation case communicating with the air supply portion and accommodating the powder inside and a mesh provided in an opening of the accommodation case, and
the accommodation case and the mesh are configured to diffuse the powder accommodated inside into the gas with the gas sent from the air supply portion and to discharge a powdery fluid that contains the powder diffused into the gas through the opening.

4. The powder feeder according to claim 1, wherein
the accommodation portion, the diffusion portion, and the discharge portion are defined by an accommodation case that accommodates the powder inside, and include a gas inlet provided in a first wall surface of the accommodation case and communicating with the air supply portion and a discharge hole provided in a second wall surface of the accommodation case different from the first wall surface as a powdery fluid outlet, and
the accommodation case is configured to diffuse the powder into the gas with the gas entering through the gas inlet and to discharge a powdery fluid that contains the powder diffused into the gas through the discharge hole.

5. The powder feeder according to claim 1, wherein
the accommodation portion and the diffusion portion define a first chamber that includes a gas inlet communicating with the air supply portion, a powdery fluid outlet communicating with the discharge portion, and a powder accommodation space accommodating the powder, the first chamber diffusing the powder into the gas with the gas blown from the gas inlet, and discharging a powdery fluid that contains the powder diffused into the gas through the powdery fluid outlet, and
the discharge portion includes a discharge port facing an inside of the living body, a powdery fluid inlet communicating with the powdery fluid outlet, and a second chamber located between the discharge port and the powdery fluid inlet and smaller in volume than the powder accommodation space.

6. The powder feeder according to claim 1, further comprising
a vibrating device that vibrates the powder accommodated in the accommodation portion.

7. The powder feeder according to claim 1, wherein
the accommodation portion includes a first accommodation portion that accommodates a first powder and a second accommodation portion that accommodates a second powder,
the air supply portion is configured to be able to supply the gas to the first accommodation portion and the second accommodation portion individually and independently, and
the diffusion portion and the discharge portion are configured to discharge a first powdery fluid obtained by diffusing the first powder into the gas when the gas is supplied to the first accommodation portion, and are configured to discharge a second powdery fluid obtained by diffusing the second powder into the gas when the gas is supplied to the second accommodation portion.

8. An endoscope comprising:
an endoscope main body including an insertion portion to be inserted into a living body;
a powder feeder to be attached to a distal end portion of the insertion portion; and
an air supply device that sends a gas to the powder feeder, wherein
the powder feeder includes
an attachment portion configured to be attached to the distal end portion of the insertion portion,
an air supply portion configured to take in the gas from an air supply nozzle of the endoscope main body,
an accommodation portion communicating with the air supply portion and configured to accommodate powder,
a diffusion portion communicating with the accommodation portion and configured to diffuse the powder accommodated in the accommodation portion into the gas fed from the air supply nozzle, and
a discharge portion communicating with the diffusion portion and configured to discharge the gas into which the powder has been diffused into the living body together with the powder, and
the air supply device applies vibration to the powder in the powder feeder due to a change in pressure of the gas when discharging the powder from the powder feeder.

9. A method for using a powder feeder, the method comprising:
inserting an insertion portion of an endoscope into a living body, extracting the insertion portion inserted into the living body from the living body, replacing a hood at a distal end portion of the insertion portion with a powder feeder accommodating powder, and connecting the powder feeder to a tube for air supply;
inserting the insertion portion to which the powder feeder is attached toward a specific part in the living body; and
sending a gas to the powder feeder located in the living body, and discharging the powder accommodated in the powder feeder to the specific part as a powdery fluid.

10. The powder feeder according to claim 2, further comprising
a vibrating device that vibrates the powder accommodated in the accommodation portion.

11. The powder feeder according to claim 3, further comprising
a vibrating device that vibrates the powder accommodated in the accommodation portion.

12. The powder feeder according to claim 4, further comprising
a vibrating device that vibrates the powder accommodated in the accommodation portion.

13. The powder feeder according to claim 5, further comprising
a vibrating device that vibrates the powder accommodated in the accommodation portion.

14. The powder feeder according to claim 2, wherein
the accommodation portion includes a first accommodation portion that accommodates a first powder and a second accommodation portion that accommodates a second powder,
the air supply portion is configured to be able to supply the gas to the first accommodation portion and the second accommodation portion individually and independently, and
the diffusion portion and the discharge portion are configured to discharge a first powdery fluid obtained by diffusing the first powder into the gas when the gas is supplied to the first accommodation portion, and are configured to discharge a second powdery fluid obtained by diffusing the second powder into the gas when the gas is supplied to the second accommodation portion.

15. The powder feeder according to claim 3, wherein
the accommodation portion includes a first accommodation portion that accommodates a first powder and a second accommodation portion that accommodates a second powder,
the air supply portion is configured to be able to supply the gas to the first accommodation portion and the second accommodation portion individually and independently, and
the diffusion portion and the discharge portion are configured to discharge a first powdery fluid obtained by diffusing the first powder into the gas when the gas is supplied to the first accommodation portion, and are configured to discharge a second powdery fluid obtained by diffusing the second powder into the gas when the gas is supplied to the second accommodation portion.

16. The powder feeder according to claim 4, wherein
the accommodation portion includes a first accommodation portion that accommodates a first powder and a second accommodation portion that accommodates a second powder,
the air supply portion is configured to be able to supply the gas to the first accommodation portion and the second accommodation portion individually and independently, and
the diffusion portion and the discharge portion are configured to discharge a first powdery fluid obtained by diffusing the first powder into the gas when the gas is supplied to the first accommodation portion, and are configured to discharge a second powdery fluid obtained by diffusing the second powder into the gas when the gas is supplied to the second accommodation portion.

17. The powder feeder according to claim 5, wherein
the accommodation portion includes a first accommodation portion that accommodates a first powder and a second accommodation portion that accommodates a second powder,
the air supply portion is configured to be able to supply the gas to the first accommodation portion and the second accommodation portion individually and independently, and
the diffusion portion and the discharge portion are configured to discharge a first powdery fluid obtained by diffusing the first powder into the gas when the gas is supplied to the first accommodation portion, and are configured to discharge a second powdery fluid obtained by diffusing the second powder into the gas when the gas is supplied to the second accommodation portion.
